(19) United States
(12) Patent Application Publication
(10) Pub. No.: US 2023/0170081 A1
(43) Pub. Date: Jun. 1, 2023

(54) MEDICAL SUPPORT APPARATUS AND MEDICAL SUPPORT SYSTEM

(71) Applicant: CANON MEDICAL SYSTEMS CORPORATION, Otawara-shi (JP)

(72) Inventors: Yohei KAMINAGA, Otawara-shi (JP); Kouji OTA, Nasushiobara (JP); Yoshifumi YAMAGATA, Sakura (JP)

(73) Assignee: CANON MEDICAL SYSTEMS CORPORATION, Otawara-shi (JP)

(21) Appl. No.: 18/058,824

(22) Filed: Nov. 25, 2022

(30) Foreign Application Priority Data
Nov. 26, 2021 (JP) 2021-192514

(51) Int. Cl. G16H 40/20; G16H 10/20

(52) U.S. Cl. CPC G16H 40/20; G16H 10/20

(57) ABSTRACT

The medical support apparatus according to the present embodiment comprises processing circuit configured to acquire interview information related to an adverse event predicted for a patient and supply the interview information to a patient terminal; and receive an interview result information, which is a patient's response result to the interview information, and evaluate a patient's adverse event based on the interview result information.

### CROSS REFERENCE TO RELATED APPLICATIONS

This application is based upon and claims the benefit of priority from the prior Japanese Patent Application No. 2021-192514, filed on Nov. 26, 2021, the entire content of which are incorporated herein by reference.

### FIELD

The embodiments disclosed in the present specification and drawings relate to a medical support apparatus and a medical support system.

### BACKGROUND

Generally, treatments for a patient initially determines a treatment plan and is made based on the determined treatment plan. Especially, in cancer treatments, it is important to continue the initially determined treatment plan until the end because having rest periods will diminish treatment effects. However, there are cases where the treatment plan of the cancer treatment is paused or stopped due to various causes such as pain during treatment or treatment progress, or fears against radiotherapy, which is one type of cancer treatment. The treatment plan being paused or stopped is also caused by a lack of opportunity of supportive communication (opportunity to talk with a doctor or nurse).

In recent years, medical support systems such as a doctor consulting system or an initial interview system that allows to receive a doctor's diagnosis remotely are being spread. Due to the spread of such medical support systems, doctors became able to appropriately treat a patient's incident outside a hospital that requires a treatment prognosis follow-up, creating opportunity of supportive communication. On the other hand, due to the spread of such medical support systems, doctors are compelled to respond to the patient's incident outside the hospital each time, which generates a problem that increases a doctor's workload.

An automatic response system that automatically responds to consultation of patients may be used to resolve the problem that increases the doctor's workload. However, conventional automatic response systems are unable to make responses that reflect a patient's individual treatment progress for a patient's consultation, since conventional automatic response systems are response based on typical fixed phrases or guidelines. This sort of problem similarly occurs not only cancer treatments but also for various treatments that require a treatment prognosis follow-up. For this reason, it is desired for the doctor to respond to a patient's incident outside the hospital in addition to minimally suppressing an increase of the doctor's workload, along with making responses that reflect the patient's individual treatment progress by the medical support apparatus. That is to say, it is desired for the doctor to diagnose the patient efficiently and appropriately.

### BRIEF DESCRIPTION OF THE DRAWINGS

FIG. 1 is a block diagram that illustrates an exemplary configuration of a medical support system according to one embodiment.

FIG. 2 is a block diagram that illustrates another exemplary configuration of the medical support system according to one embodiment.

FIG. 3 is a block diagram that illustrates a configuration example of a medical support apparatus according to one embodiment.

FIG. 4 is a flowchart that describes a content of a notification process executed in the medical support apparatus according to one embodiment.

FIG. 5 is a flowchart that describes a content of the notification process executed in the medical support apparatus according to one embodiment.

FIG. 6 is a diagram that illustrates an exemplary question list LT1 to inquire a patient condition according to one embodiment.

FIG. 7 is a diagram that illustrates an exemplary display status of a display of a patient terminal according to one embodiment.

FIG. 8 is a diagram that illustrates an exemplary display status of the display of the patient terminal according to one embodiment.

FIG. 9 is a diagram that illustrates an exemplary display status of the display of the patient terminal according to one embodiment.

FIG. 10 is a diagram that illustrates an exemplary display status of a display of the medical support apparatus according to one embodiment.

FIG. 11 is a diagram that illustrates an exemplary display status of the display of the patient terminal according to one embodiment.

FIG. 12 is a flowchart that describes a content of a re-inquiry process executed in the medical support apparatus according to one embodiment.

FIG. 13 is a diagram that illustrates an exemplary display status of the display of the patient terminal according to one embodiment.

FIG. 14 is a diagram that illustrates an exemplary display status of the display of the patient terminal according to one embodiment.

FIG. 15 is a flowchart that describes a content of a first update process executed in the medical support apparatus according to one embodiment.

FIG. 16 is a flowchart that describes a content of a second update process executed in the medical support apparatus according to one embodiment.

FIG. 17 is a flowchart that describes a content of a summary creation process executed in the medical support apparatus according to one embodiment.

FIG. 18 is a diagram that illustrates an exemplary display status of a summary and an inspection order information displayed on the display of the medical support apparatus according to one embodiment.

### DETAILED DESCRIPTION

With reference to the drawings below, embodiments of a medical support apparatus and a medical support system will be described. Note that, in the description below, same reference signs are given for components substantially identical in terms of configuration and function, and duplicate description will be given only when necessary.

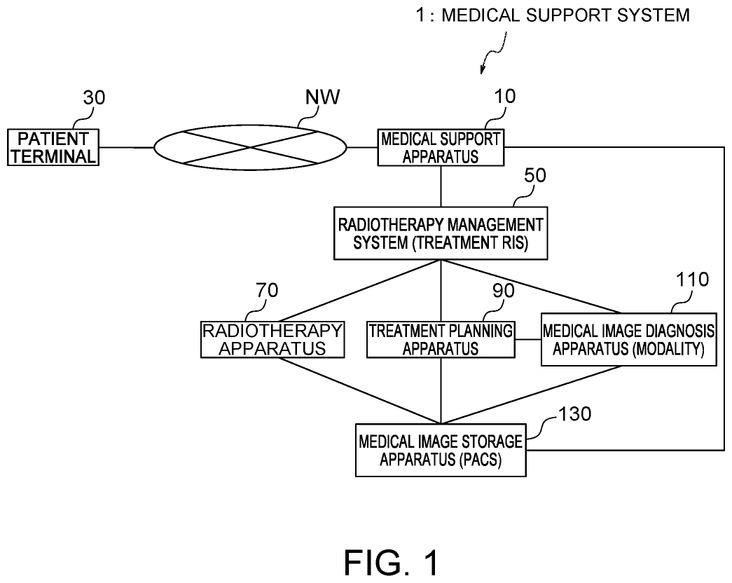

FIG. 1 is a block diagram that illustrates an exemplary configuration of a medical support system 1 according to a present embodiment. As shown in FIG. 1, the medical support system 1 comprises a medical support apparatus 10, a patient terminal 30, a radiotherapy management system (treatment radiology information system (RIS)) 50, a radiotherapy apparatus 70, a treatment planning apparatus 90, a medical image diagnosis apparatus (modality) 110, and a medical image storage apparatus 130. The medical support apparatus 10 and the patient terminal 30 are connected to each other in a communicative manner via a network NW by a public line such as an internet. Likewise, the medical support apparatus 10, the radiotherapy management system 50, the radiotherapy apparatus 70, the treatment planning apparatus 90, the medical image diagnosis apparatus 110, and the medical image storage device 130 are connected in a communicative manner via an in-hospital network by private line in a hospital.

Note that the medical support apparatus 10, the patient terminal 30, the radiotherapy management system 50, the radiotherapy apparatus 70, the treatment planning apparatus 90, the medical image diagnosis apparatus 110, and the medical image storage device 130 may be connected to each other in a communicative manner via the network NW by the public line such as the internet.

The medical support apparatus 10 is an apparatus that evaluates a patient's adverse event or determines how to handle a patient based on a patient's medical information. For instance, in the present embodiment, the medical support apparatus 10 acquires the patient's medical information from the radiotherapy management system 50 or the medical image storage apparatus 130 etc.; predicts the patient's adverse event based on the acquired patient's medical information; acquires an interview information related to the adverse event predicted for the patient; supplies the interview information to the patient terminal 30 as an inquiry about the patient condition; receives an interview result information from the patient terminal 30 as the patient's response to the inquiry, and evaluates the patient's adverse event according to the patient condition based on the interview result information. Likewise, in the present embodiment, the medical support apparatus 10 determines how to handle the patient based on an evaluation result of the patient's adverse event. For instance, in the example of FIG. 1, the medical support apparatus 10 is configured to be combined with the doctor system operated by the doctor. Likewise, when the medical support apparatus 10 is combined with the doctor system, the medical support apparatus 10, for instance, may be an electronic medical chart system.

Note that the medical support apparatus 10 acquires the patient's medical information from the radiotherapy management system 50 or the medical image storage apparatus 130, but the medical support apparatus 10 may store the patient's medical information and acquire the stored medical information.

Here, the medical information refers to information related to medical care such as an examination, a diagnosis, or a treatment; an inspection, a checkup, or treatment plans that include an executed treatment plan and a non-executed treatment plan. Note that the medical information may not include every information related to the medical care, the inspection, the checkup, and the treatment plans, but is sufficient to include at least any information. Likewise, the medical information may include information other than the information related to the medical care, the inspection, the checkup, and the treatment plan.

Likewise, the adverse event indicates all sorts of unfavorable medical events that occurs to the patient. An aggravation of the patient condition caused by a disorder or an occurrence of a side effect from treatments are included in the adverse event. For instance, the aggravation of the patient condition caused by a disorder may be a heart stroke or an apoplexy etc. Likewise, the occurrence of the side effect from treatments includes, for instance, bone marrow suppression skin disorder, digestive insufficiency, hypersensitivity, or renal dysfunction from radiotherapy, or liver failure or renal dysfunction from medication.

Likewise, the interview information refers to information for interviewing the patient. In the present embodiment, the interview information, for instance, refers to questions for the patient.

Likewise, the interview result information refers to a response result to the interview information. In the present embodiment, the interview result information, for instance, includes the patient's response result such as a patient's current condition to the questions about the adverse event.

The patient terminal 30 refers to a terminal used by the patient. The patient terminal 30, for instance, in the present embodiment, receives the interview information from the medical support apparatus 10 by receiving the interview information related to the adverse event predicted for the patient as the questions related to an inquiry from the medical support apparatus 10 via the network NW. The patient terminal 30 also receives the patient's response to the received interview information. The patient terminal 30 also supplies to the medical support apparatus 10 the interview result information for the interview information by sending the patient's response result to the received interview information to the medical support apparatus 10 via the network NW. The patient terminal 30, for instance, refers to a portable terminal such as a smartphone or a tablet personal computer (PC) held by the patient or a family of the patient, or a terminal such as a PC. The patient terminal also may be a wearable terminal which the patient equips on own's body.

Note that, although one patient terminal 30 is connected to the network NW in the example of FIG. 1 according to the present embodiment, a number of the patient terminals connected to the network NW is not limited to this. That is to say, the number of the patient terminal 30 is arbitrary, and a plurality of patient terminals 30 may be connected to the network NW.

The radiotherapy management system 50 refers to an apparatus for managing various information to perform radiotherapy. In the present embodiment. the radiotherapy management system 50, for instance, manages medical information such as a medical care information to perform radiotherapy or a treatment plan information, sent from the radiotherapy apparatus 70, the treatment planning apparatus 90, or the medical image diagnosis apparatus 110 etc. via the in-hospital network.

The radiotherapy apparatus 70 refers to an apparatus that performs radiotherapy by irradiating radiation to the patient. The radiotherapy apparatus 70, for instance, according to the present embodiment, performs radiotherapy following the treatment plan of the treatment planning apparatus 90 under a management of the radiotherapy management system 50.

The treatment planning apparatus 90 refers to an apparatus that makes a radiotherapy plan by the radiotherapy apparatus 70, using a medical image captured by the medical image diagnosis apparatus 110. Specifically, for instance, the treatment planning apparatus 90 uses the medical image captured by the medical image diagnosis apparatus 110 to identify a position of a treatment target such as a tumor inside the patient, and makes the plan such as a dose of radiation, an irradiation angle, or a frequency of irradiation for which the radiotherapy apparatus 70 will irradiate to the tumor which the position is identified using the medical image.

The medical image diagnosis apparatus 110 refers to an apparatus that collects medical images by imaging the patient. The medical image diagnosis apparatus 110 sends the imaged medical image to the radiotherapy management system 50, the treatment planning apparatus 90, or the medical image storage apparatus 130 etc. via the in-hospital network. For instance, the medical image diagnosis apparatus 110 refers to an X-ray imaging apparatus such as an X-ray diagnosis apparatus or an X-ray Computed Tomography (CT) apparatus, an ultrasonic diagnosis apparatus, or a Magnetic Resonance Imaging (MRI) apparatus. Note that the medical image diagnosis apparatus 110 is also called as a modality.

The medical image storage apparatus 130 refers to an apparatus that stores the medical images. According to the present embodiment, the medical image storage apparatus 130, for instance, stores the medical images imaged by the medical image diagnosis apparatus 110. Likewise, the medical image storage apparatus 130 sends the stored medical images etc. to the medical support apparatus 10 or the treatment planning apparatus 90 via the in-hospital network. The medical image storage apparatus 130, for instance, refers to an image server such as a Picture Archiving and Communication System (PACS).

The network NW refers to a general information network that uses telecommunication technology. A typical example of the network NW is the internet. That is to say, the network NW is the public line network which connects between a house of the patient and the hospital. On the other hand, the in-hospital network, for instance, refers to a network configured by a wired/wireless Local Area Network (LAN) such as a backbone LAN of the hospital, a telecommunication network, an optical fiber communication network, or the private line such as a cable communication network and a satellite communication network.

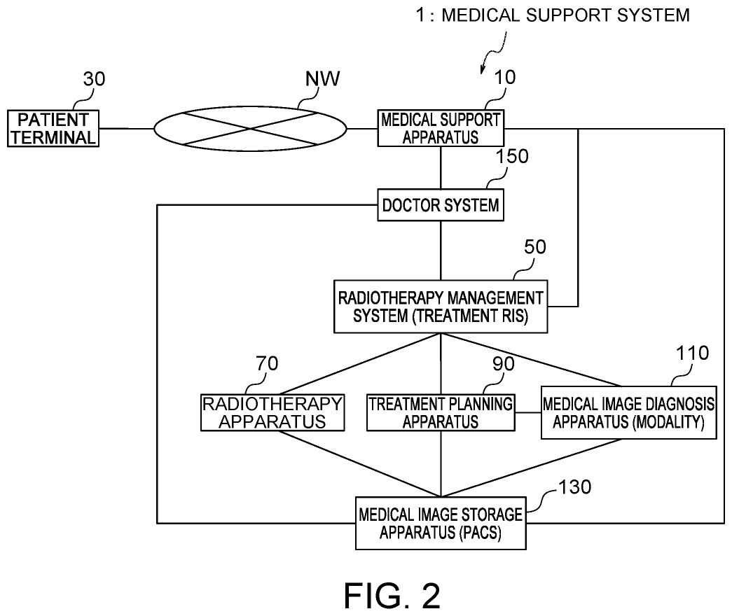

Note that the medical support apparatus 10 in the medical support system 1 according to the present embodiment described above is combined with the doctor system, but the doctor system does not necessarily have to be combined with the medical support apparatus 10. That is to say, the doctor system may be separated from the medical support apparatus 10. FIG. 2 is a block diagram that illustrates another exemplary configuration of the medical support system 1 according to the present embodiment for a case where the doctor system and the medical support apparatus 10 are separated, and is a diagram that corresponds to FIG. 1. The detailed description different from that of FIG. 1 described above will be made below.

As shown in FIG. 2, the medical support system 1 according to the present embodiment comprises the medical support apparatus 10, the patient terminal 30, the radiotherapy management system 50, the radiotherapy apparatus 70, the treatment planning apparatus 90, the medical image diagnosis apparatus 110, the medical image storage apparatus 130, and the doctor system 150. Note that the exemplary configuration of the patient terminal 30, the radiotherapy management system 50, the radiotherapy apparatus 70, the treatment planning apparatus 90, the medical image diagnosis apparatus 110, and the medical image storage apparatus 130 will be omitted since this is similar to that of FIG. 1 described above.

Similar to the medical support apparatus 10 shown in FIG. 1, the medical support apparatus 10 shown in FIG. 2 refers to an apparatus that evaluates the patient's adverse event or determines how to handle the patient based on the patient's medical information. For instance, in the present embodiment, the medical support apparatus 10 acquires the patient's medical information from the radiotherapy management system 50, the medical image storage apparatus 130, or the doctor system 150; predicts the patient's adverse event based on the acquired patient's medical information; acquires the interview information related to the adverse event predicted for the patient; supplies the interview information to the patient terminal 30 as the inquiry about the patient condition; receives from the patient terminal 30 the interview result information as the patient's response to the questions; and evaluates the patient's adverse event according to the patient condition based on the interview result information. In the present embodiment, the medical support apparatus 10 also determines how to handle the patient based on the evaluation result of the patient's adverse event. The medical support apparatus 10, for instance, refers to a terminal such as a computer.

The doctor system 150 refers to a system operated by the doctor. The doctor system 150, in the present embodiment, for instance, refers to a terminal such as the computer included the doctor system 150. The doctor system 150 acquires the medical information such as the diagnosis information or the treatment plan information from the radiotherapy management system 50 or the medical image storage apparatus 130, and the doctor diagnoses based on the acquired medical information. The doctor system 150, for instance, may be an electronic medical chart system.

Note that, in the example of FIG. 2 according to the present embodiment, although one doctor system 150 is connected to the medical support apparatus 10, the radiotherapy management system 50, or the medical image storage apparatus 130, the number of the doctor system 150 connected to the medical support apparatus 10, the radiotherapy management system 50, or the medical image storage apparatus 130 is not limited to this. That is to say, the number of the doctor system 150 is arbitrary, and a plurality of doctor systems may be connected to the medical support apparatus 10, the radiotherapy management system 50, or the medical image storage apparatus 130.

Likewise, in the example of FIG. 2 according to the present embodiment, although the medical support apparatus 10 is not only connected to the doctor system 150 but also to the radiotherapy management system 50 and the medical image storage apparatus 130, the medical support apparatus 10 may not be connected to the radiotherapy management system 50 and the medical image storage apparatus 130. That is to say, the medical support apparatus 10 may acquire the patient's medical information from the radiotherapy management system 50 and the medical image storage apparatus 130 via the doctor system 150.

As shown in FIGS. 1 and 2, the medical support system 1 according to the present embodiment may not only be applied where the medical support apparatus 10 and the doctor system 150 are combined but also where the medical support apparatus 10 and the doctor system 150 are separate. The detailed description of the present embodiment will be described below with an example where the medical support apparatus 10 and the doctor system 150 are combined.

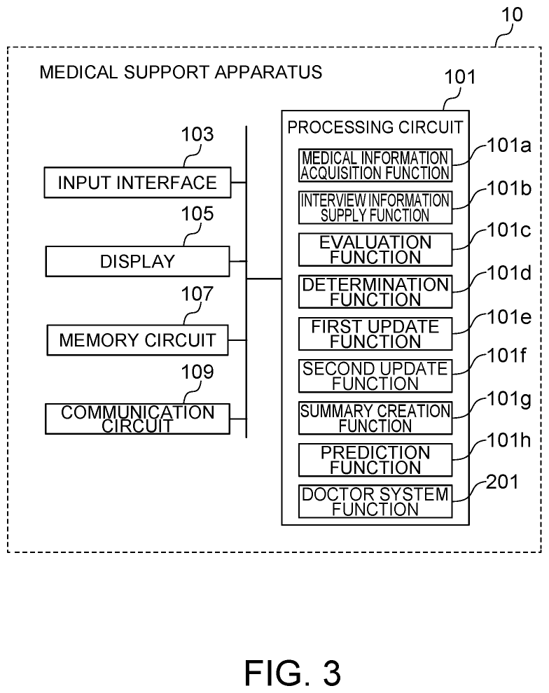

FIG. 3 is a block diagram that illustrates a configuration example of the medical support apparatus 10 according to the present embodiment. As shown in FIG. 3, the medical support apparatus 10 comprises a processing circuit 101, an input interface 103, a display 105, a memory circuit 107, and a communication circuit 109. The plurality of elements to configure the medical support apparatus 10 may be stored in one housing or separately stored in a plurality of housings.

The processing circuit 101 refers to an arithmetic circuit that performs various operations. The processing circuit 101 according to the present embodiment, for instance, supplies the interview information to the patient, evaluates the patient's adverse event based on the interview result information for the interview information, or determines how to handle the patient based on the evaluation result of the patient's adverse event.

For this reason, the processing circuit 101 according to the present embodiment has a medical information acquisition function 101a, an interview information supply function 101b, an evaluation function 101c, a determination function 101d, a first update function 101e, a second update function 101f, a summary creation function 101g, a prediction function 101h, and a doctor system function 201. The medical information acquisition function 101a is equivalent to a medical information acquirer in the present embodiment; the interview information supply function 101b is equivalent to an interview information supplier in the present embodiment; the evaluation function 101c is equivalent to an evaluator in the present embodiment; the determination function 101d is equivalent to a determiner in the present embodiment; the first update function 101e is equivalent to a first updater in the present embodiment; the second update function 101f is equivalent to a second updater in the present embodiment; the summary creation function 101g is equivalent to a summary creator in the present embodiment; the prediction function 101h is equivalent to a predictor in the present embodiment; and the doctor system function 201 is equivalent to a doctor system unit in the present embodiment.

In the embodiment shown in FIG. 3, each processing function executed in the medical information acquisition function 101a, the interview information supply function 101b, the evaluation function 101c, the determination function 101d, the first update function 101e, the second update function 101f, the summary creation function 101g, the prediction function 101h, and the doctor system function 201 is stored in the memory circuit 107 in a form of a computer executable program. The processing circuit 101 refers to a processor that realizes functions corresponding to each program by reading and executing the program from the memory circuit 107. In other words, the processing circuit 101 in a state that has read each program has each function shown in the processing circuit 101 of FIG. 3. Note that, in FIG. 3, it was explained as the medical information acquisition function 101a, the interview information supply function 101b, the evaluation function 101c, the determination function 101d, the first update function 101e, the second update function 101f, the summary creation function 101g, the prediction function 101h, and the doctor system function 201 being realized in a single processing circuit, but these functions may be realized by combining a plurality of independent processors to configure the processing circuit 101 and have each processor execute the program.

The input interface 103, for instance, receives various input operations from the doctor, converts the received input operations into an electrical signal, and outputs to the processing circuit 101. The input interface 103, for instance, is realized by a mouse, a keyboard, a trackball, a manual switch, a foot switch, a button, or a joystick etc.

The display 105 displays various information. For instance, the display 105 displays such as a Graphical User Interface (GUI) to receive various operations from a user. In the present embodiment, the display 105, for instance, may be configured by a liquid crystal display or a Cathode Ray Tube (CRT) display.

The memory circuit 107, for instance, is realized by a Random Access Memory (RAM), a semiconductor memory element such as a flash memory, a hard disk, or an optical disk etc. In the present embodiment, the memory circuit 107, for instance, stores various information such as information related to the adverse event, questions for the patient related with the adverse event, and the interview result information which is the patient's response result to the questions. Note that the memory circuit 107 may store the patient's medical information. The memory circuit 107 is equivalent to the memory according to the present embodiment. Also, the questions are an example of the interview information according to the present embodiment.

Here, the information related to the adverse event includes the adverse event, information related to symptoms associated with the adverse event, and information related to a time when the adverse event will occur. The information related to symptoms associated with the adverse event refers to information of symptoms attributed to the adverse event. The information related to symptoms, for instance, refers to fever, weariness, or a pain of the body. The information related to the time when the adverse event will occur, for instance, refers to information of time when the adverse event will occur after treatment. For instance, when the adverse event is bone marrow suppression or skin disorder, the information of time when the adverse event will occur is during treatment or right after treatment, and when the adverse event is renal dysfunction, the information of time when the adverse event will occur is around 3 months after treatment. Note that the information related to the adverse event may not include information related to the time when the adverse event will occur.

The communication circuit 109 implements various protocols for telecommunication in response to a form of the network NW or the in-hospital network. The communication circuit 109 realizes communications with other apparatuses via the network NW or the in-hospital network following the various protocols. In the present embodiment, the communication with the patient terminal 30 is realized by the medical support apparatus 10 being connected to the network NW via the communication circuit 109. Likewise, in the present embodiment, the communication with the radiotherapy management system 50 or the medical image storage apparatus 130 is realized by the medical support apparatus 10 being connected to the in-hospital network via the communication circuit 109.

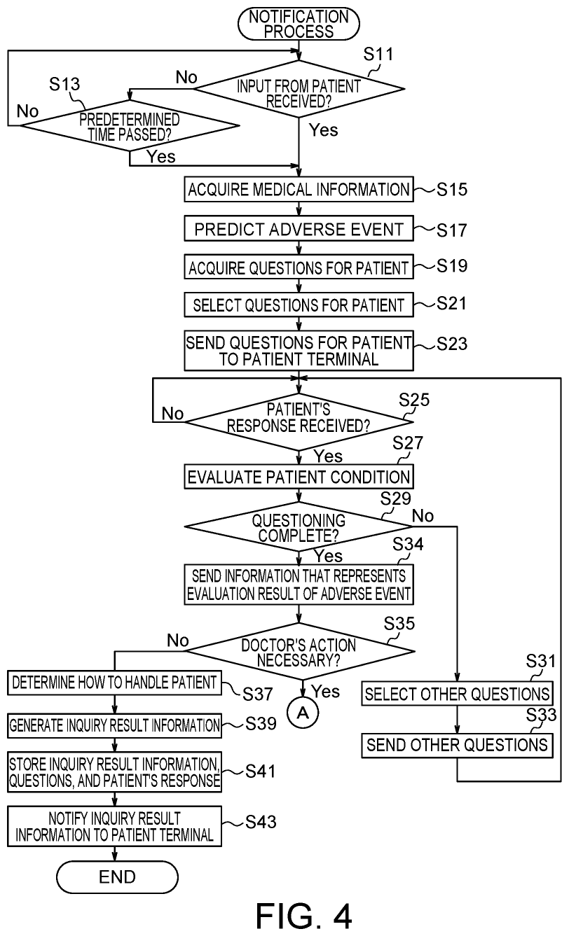

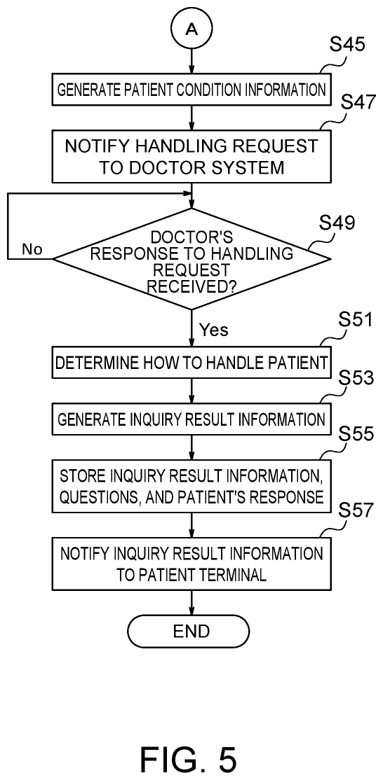

FIGS. 4 and 5 are a flowcharts that describe a content of a notification process executed in the medical support apparatus 10 according to the present embodiment. In the notification process, the medical support apparatus 10 predicts the patient's adverse event based on the patient's medical information; supplies the questions to the patient terminal 30 to inquire the patient; evaluate the patient's adverse event according to the patient condition based on the interview result information, which is the patient's response result to the questions; or determine how to handle the patient based on the evaluation result of the patient's adverse event and notify the patient terminal 30. For instance, the notification process refers to a process executed when an input from the patient about the patient condition has been received or when a predetermined period has passed from a previous medical care.

As shown in FIG. 4, the medical support apparatus 10 first distinguishes whether the input from the patient has been received or not (Step S11). The process of distinguishing whether the input from the patient has been received or not is realized by the interview information supply function 101b in the processing circuit 101. Specifically, the medical support apparatus 10 distinguishes whether the input related to the patient condition sent by the patient from the patient terminal 30 via the communication circuit 109 has been received or not. That is to say, the medical support apparatus 10 distinguishes whether a request from the patient terminal 30 via the communication circuit 109 has been received or not.

Then, when the input from the patient has not been received, i.e., when the request from the patient terminal 30 has not been received (Step S11: No), the medical support apparatus 10 distinguishes whether the predetermined period has passed or not from the previous medical care (Step S13). The process of distinguishing whether the predetermined period has passed or not from the previous medical care is realized by the interview information supply function 101b in the processing circuit 101. Specifically, the medical support apparatus 10 distinguishes whether the predetermined period has passed or not from the previous medical care, i.e., whether the predetermined time from the previous medical care has arrived or not. Then, if the predetermined period has not passed from the previous medical care, i.e., if the predetermined time has not passed from the previous medical care, the medical support apparatus 10 standbys repeating the process of Step S11 and Step S13 until receiving from the patient the input related to the patient condition or until the predetermined period passes from the previous medical care.

On the other hand, at Step S11 described above, when the input related to the patient condition has been received from the patient, i.e., when the request from the patient terminal 30 has been received (Step S11: Yes), or when the predetermined period has passed from the previous medical care, i.e., when the predetermined time has arrived (Step S13: Yes), the medical support apparatus 10 acquires the patient's medical information (Step S15). The process of acquiring the patient's medical information is realized by the medical information acquisition function 101a in the processing circuit 101. Specifically, the medical support apparatus 10 acquires the patient's medical information stored in the memory circuit 107 via the communication circuit 109.

Note that the patient's medical information is not limited to be acquired from the memory circuit 107 by the medical information acquisition function 101a in the processing circuit 101, but may be directly acquired from the radiotherapy management system 50 or the medical image storage apparatus 130 by the medical information acquisition function 101a in the processing circuit 101 via the communication circuit 109, or may once store the acquired patient's medical information to the memory circuit 107 and then acquire from the memory circuit 107 by the medical information acquisition function 101a in the processing circuit 101.

Next, as shown in FIG. 4, the medical support apparatus 10 predicts the adverse event (Step S17). The process of predicting the adverse event is realized by the prediction function 101h in the processing circuit 101. Specifically, the medical support apparatus 10 predicts the patient's one or more adverse event from the information related to the adverse event stored in the memory circuit 107 based on the patient's medical event acquired at Step S15.

For instance, when “radiotherapy to head and neck,” which is one type of cancer treatment, is performed as the patient's medical information, bone marrow suppression, skin disorder, digestive insufficiency, or renal dysfunction etc., are predicted from the information related to the adverse event. Note that, when the input from the patient related to the patient condition has been received as the request from the patient terminal at Step S11, the patient's adverse event may be predicted based on the input from the patient received at Step S11 and the patient's medical information. That is to say, when a plurality of adverse events are predicted based on the patient's medical information at Step S17, the plurality of adverse events may be further narrowed down based on the input from the patient received at Step S11.

At Step S17, the medical support apparatus 10 may also predict the patient's adverse event based on a time of receiving the input from the patient at Step S11 or a time after a passage of the predetermined period at Step S13, i.e., the time of receiving the request from the patient terminal 30 at Step S11 or the predetermined time at Step S13, and the patient's medical information. For instance, when “radiotherapy to head and neck,” which is one type of cancer treatment, is performed as the patient's medical information, and when the time of receiving the request from the patient terminal 30 or the predetermined time is right after “radiotherapy to head and neck,” the medical support apparatus 10 may predict bone marrow suppression or skin disorder etc., based on the information related to a time when the adverse event according to the information related to the adverse event appears. That is to say, the medical support apparatus 10 may predict the adverse event in response to the time of receiving the request from the patient terminal 30 or the predetermined time.

Next, as shown in FIG. 4, the medical support apparatus 10 acquires the questions for the patient (Step S19). The process of acquiring the questions for the patient is realized by the interview information supply function 101b in the processing circuit 101. Specifically, the medical support apparatus 10 acquires from the memory circuit 107 the interview information related to the adverse event predicted for the patient as the questions for the patient based on the patient's medical information. More specifically, for instance, the medical support apparatus 10 acquires from a question list stored in the memory circuit 107 the questions related to the adverse event predicted for the patient at Step S17 based on the patient's medical information. For instance, when bone marrow suppression and renal dysfunction are predicted as the adverse events at Step S17, the medical support apparatus 10 acquires the questions related to bone marrow suppression and renal dysfunction from the question list.

Note that, when the input from the patient related to the patient condition has been received at Step S11, the medical support apparatus 10 may, at Step S19, acquire the questions based on the input by the patient received at Step S11 from the question list stored in the memory circuit 107 as well as the questions related to the adverse event predicted for the patient at Step S17. More specifically, for instance, when the input from the patient such as “I can't stop coughing” has been received as the input from the patient related to the patient condition at Step S11, the medical support apparatus 10 may acquire questions related to coughing from the question list stored in the memory circuit 107.

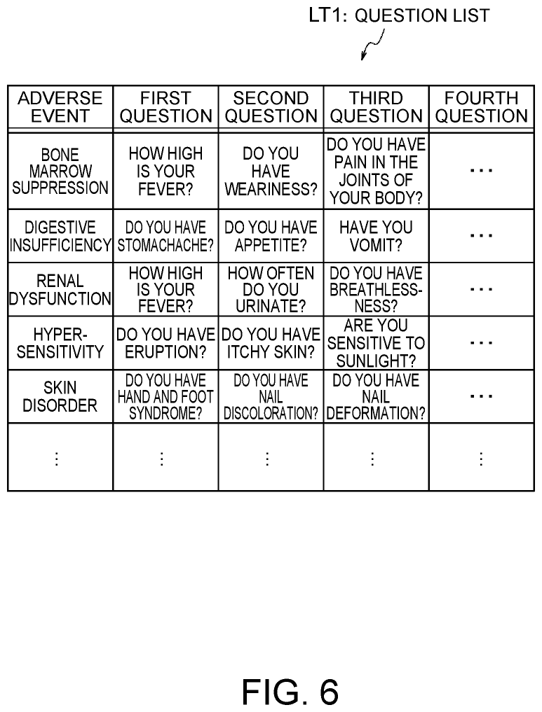

FIG. 6 is a diagram that illustrates an exemplary question list LT1 to inquire a patient condition according to the present embodiment. As shown in FIG. 6, in the present embodiment, the question list LT1 includes the adverse event and the questions related to the adverse event. For instance, with an example of bone marrow, which is one adverse event, “How high is your fever??” is set as a first question for bone marrow, “Do you have weariness?” is set as a second question for bone marrow, and “Do you have pain in the joints of your body?” is set as a third question for bone marrow.

Note that three questions from the first question to the third question are set for each adverse event in the question list LT1 shown in FIG. 6, but the number of questions to be set is arbitrary. That is to say, the number of questions set for one adverse event may be 2 or less, or 4 or more. Likewise, the number of questions set for each adverse event may be different.

Likewise, the question list LT1 in the medical support apparatus 10 may be set arbitrarily, and the questions may be added or modified in response to a preference of the doctor or by an arbitrary setting of the doctor. When the doctor sets an arbitrary question, the question set by the doctor is also stored in the memory circuit 107 as the question list LT1.

The question list LT1 shown in FIG. 6 further includes the adverse event and the questions related to the adverse event, but may be set to include the arbitrary questions unrelated with the adverse event. That is to say, the questions included in the question list LT1 is arbitrary. For instance, the question list LT1 may include questions related to coughing, questions related to wheezing, or questions related to eruption with item of another adverse event in the question list LT1.

Next, as shown in FIG. 4, the medical support apparatus 10 selects the questions for the patient (Step S21). The process of selecting the questions for the patient is realized by the interview information supply function 101b in the processing circuit 101. Specifically, the medical support apparatus 10 selects the questions for the patient from the questions acquired at Step S19.

For instance, if the questions related to bone marrow suppression and renal dysfunction are acquired from the question list LT1 at Step S19, the medical support apparatus 10, as shown in FIG. 6, selects either of the second question for bone marrow “Do you have weariness?” or the second question for renal dysfunction “How often do you urinate?” as a non-common question. That is to say, if the plurality of adverse events are selected at Step 17, the medical support apparatus 10 selects a question that narrows down the plurality of adverse events. Note that the medical support apparatus 10 may select a common question related to each adverse event if the questions related to the plurality of adverse events are acquired at Step S19. For instance, as shown in FIG. 6, if the questions related to bone marrow suppression and renal dysfunction are acquired from the question list LT1 at Step S19, the medical support apparatus 10 may select “How high is your fever?” as the common question. Likewise, if bone marrow is selected as a single adverse event at Step S17, the medical support apparatus 10 may select any questions related to bone marrow.

Next, as shown in FIG. 4, the medical support apparatus 10 sends the questions for the patient to the patient terminal 30 (Step S23). The process of sending the questions for the patient to the patient terminal 30 is realized by the interview information supply function 101b in the processing circuit 101. Specifically, the medical support apparatus 10 sends via the communication circuit 109 the questions related to the adverse event predicted for the patient selected at Step S21 to inquire the patient condition. That is to say, the medical support apparatus 10 supplies to the patient terminal 30 the interview information related to the adverse event predicted for the patient via the communication circuit 109. The medical support apparatus 10 also sends the adverse event predicted for the patient at Step S17 along with sending the questions for the patient to the patient terminal 30. That is to say, the medical support apparatus 10 supplies to the patient terminal 30 the predicted adverse event along with the questions related to the adverse event predicted for the patient selected at Step S21. Note that, at Step S23, the medical support apparatus 10 may not send the adverse event predicted at Step S17.

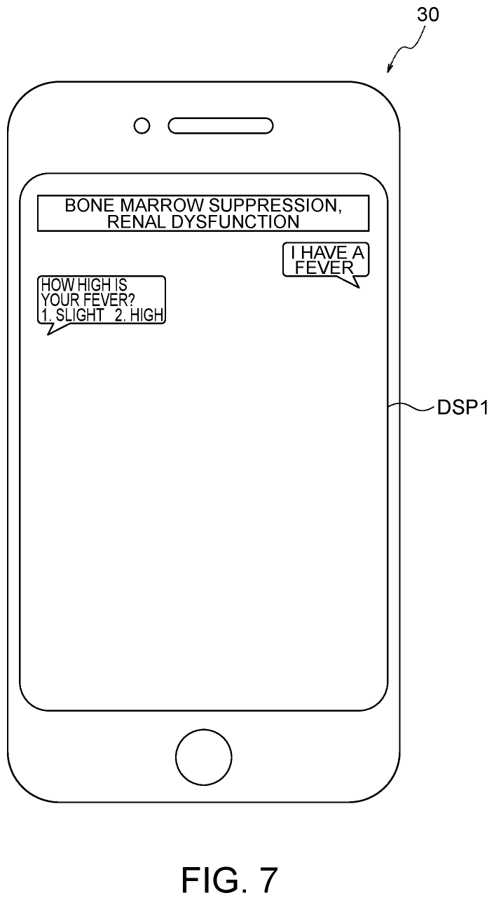

FIG. 7 is a diagram that illustrates an exemplary display status of a display DSP1 of the patient terminal 30 according to the present embodiment. As shown in FIG. 7, the patient terminal 30 receives the questions related to the adverse event predicted for the patient and the adverse event supplied by the medical support apparatus 10, and displays the questions related to the adverse event predicted for the patient and the adverse event on the display DSP1. More specifically, as shown in FIG. 7, a possibility of bone marrow and renal dysfunction is displayed as the adverse event on an upper portion of the display DSP1, “I have a fever” input by the patient via the patient terminal 30 is displayed on a right side of the display, “How high is your fever? 1. slight 2. high” is displayed on a left side of the display DSP1 as the question for the patient sent by the medical support apparatus 10.

Next, as shown in FIG. 4, the medical support apparatus 10 distinguishes whether the patient's response to the inquiry has been received or not (Step S25). The process of distinguishing whether the patient's response has been received or not is realized by the interview information supply function 101b in the processing circuit 101. Specifically, the medical support apparatus 10 distinguishes whether the patient's response to the questions sent at Step S23 has been received or not via the communication circuit 109. That is to say, the medical support apparatus 10 distinguishes whether the interview result information for the interview information supplied at Step S23 has been received or not via the communication circuit 109. Then, if the patient's response to the questions has not been received from the patient terminal 30, i.e., the interview result information for the interview information has not been received (Step S25: No), the medical support apparatus 10 standbys until receiving the patient's response to the questions from the patient terminal 30.

On the other hand, if the patient's response to the questions sent at Step S23 has been received from the patient terminal 30 at Step S25, i.e., if the interview result information for the interview information supplied at Step S23 has been received (Step S25: Yes), the medical support apparatus 10 evaluates the patient condition as shown in FIG. 4 (Step S27). The process of evaluating the patient condition is realized by the evaluation function 101c in the processing circuit 101. Specifically, the medical support apparatus 10 derives the evaluation result of the patient's adverse event by evaluating the patient's adverse event according to the patient condition based on the interview result information, which is the patient's response result received at Step S25.

More specifically, the medical support apparatus 10 evaluates the patient's adverse event so as to identify the adverse event and a severity of symptoms based on the interview result information received at Step S25. The evaluation result of the patient's adverse event, for instance, includes the adverse event and the severity of symptoms identified by evaluating the patient's adverse event. Identifying the adverse event, for instance, may be done by comparing the information related to the adverse event stored in the memory circuit 107 and the interview result information received at Step S25. If the adverse event is identified by this comparison, the medical support apparatus 10 derives the identified adverse event as the evaluation result of the patient's adverse event. On the other hand, if the adverse event is not identified by this comparison, the medical support apparatus 10 derives the evaluation result that the adverse event cannot be identified. Likewise, the severity of symptoms is identified from the interview result information received at Step S25 by evaluating whether each symptom is mild or severe. Note that the evaluation result of the patient's adverse event may include an evaluation comment which indicates whether there is a problem or not based on the severity of the identified symptoms.

Next, as shown in FIG. 4, the medical support apparatus 10 distinguishes whether the question is completed or not (Step S29). The process of distinguishing whether the question is completed or not is realized by the interview information supply function 101b in the processing circuit 101. Specifically, the medical support apparatus 10 distinguishes whether the question is completed or not based on the evaluation result of the patient's adverse event derived at Step S27. Then, if the question is not completed, the medical support apparatus 10 selects another question for the patient (Step S31). Here, being not completed, for instance, refers to a case where the adverse event or the severity of symptoms is not identified and another question may be selected.

At Step S31, the process of selecting another question is realized by the interview information supply function 101b in the processing circuit 101. Specifically, the medical support apparatus 10 selects another question related to the adverse event predicted for the patient at Step S17 from the questions acquired at Step S19.

More specifically, if there are multiple candidates of the adverse event predicted at Step S17, the medical support apparatus 10 generates the interview information that narrows down the plurality of adverse events by selecting the questions that narrow down the plurality of adverse events. For instance, if bone marrow suppression and renal dysfunction are selected as the plurality of adverse events at Step S17, the medical support apparatus 10 compares the questions for bone marrow suppression and the questions for renal dysfunction, and selects either one of the third question for bone marrow, “Do you have pain in the joints of your body?” or a third question for renal dysfunction, “Do you have breathlessness?” which are non-common questions. Likewise, if one adverse event is predicted, the medical support apparatus 10 generates the interview information by selecting another question to identify the severity of symptoms.

Note that, even when the adverse event is identified, at Step 529, based on the evaluation result of the patient's adverse event derived at Step S27, the medical support apparatus 10 may generate the interview information that identifies the severity of symptoms by selecting another question to identify the severity of symptoms. Likewise, even when the adverse event and the severity of symptoms are identified, if there are questions left in the question list LT1 that is set and supposing that the questioning is not completed, the medical support apparatus 10 may generate the interview information that narrows down the plurality of adverse events by selecting other questions from the rest of the questions.

Next, as shown in FIG. 4, the medical support apparatus 10 sends other questions for the patient selected at Step S31 (Step S33). The process of sending the other questions is realized by the interview information supply function 101b in the processing circuit 101. Specifically, the medical support apparatus 10 sends the questions related to the adverse event selected at Step S31 via the communication circuit 109 to the patient terminal 30 to inquire the patient condition. That is to say, the medical support apparatus 10 supplies the other questions to the patient terminal 30. Then, after sending to the patient terminal 30 the other questions related to the adverse event selected at Step S31 to inquire the patient condition, the medical support apparatus 10 standbys until receiving the patient's response to the inquiry. That is to say, the process of Step 25, Step S27, Step S29, Step S31, and Step S33 are repeated until the questioning is completed.

On the other hand, when the questioning is completed, at Step S29, based on the evaluation result of the patient's adverse event derived at Step S27 (Step S29: Yes), the medical support apparatus 10 sends the information that represents the evaluation result of the adverse event (Step S34). Here, the questioning being completed refers to a case where the adverse event and the severity of symptoms are identified, or when the questions for the patient acquired at Step S19 are completed and other questions cannot be selected.

At Step S34, the process of sending the information that represents the evaluation result of the adverse event is realized by the evaluation function 101c in the processing circuit 101. Specifically, the medical support apparatus 10 generates the information that represents the evaluation result of the patient's adverse event and supplies the generated information that represents the evaluation result of the patient's adverse event to the patient terminal 30 and/or the doctor system 201 by sending the generated information that represents the evaluation result of the patient's adverse event to the patient terminal 30 and/or the doctor system function 201.

Here, the doctor system function 201 allows the medical support apparatus 10 to function as the doctor system operated by the doctor. The doctor system function 201, for instance, notifies a patient's handling request via the display 105, displays the information acquired from the memory circuit 107 to the display 105, displays the information acquired via the communication circuit 109 to the display 105, or receives the electrical signal of the input operation received from the doctor via the input interface based on the information sent from the other functions in the processing circuit 101.

The information that represents the evaluation result of the patient's adverse event is information that includes the adverse event and the severity of symptoms identified based on the interview result information. Note that the information that represents the evaluation result of the patient's adverse event may include information other than the identified adverse event and the severity of symptoms.

Note that, at Step S34, the medical support apparatus 10 may select a destination of the information that includes the evaluation result of the patient's adverse event based on the evaluation result of the patient's adverse event. Specifically, the medical support apparatus 10 may supply the information that includes the evaluation result of the patient's adverse event to the patient terminal 30 if the severity of symptoms is mild, and supply the information that includes the evaluation result of the patient's adverse event to the doctor system function 201 if the severity of symptoms is severe or the adverse event cannot be identified based on the severity of symptoms according to the information that represents the evaluation result of the patient's adverse event.

The notification process may not send the information that represents the evaluation result of the patient's adverse event. If the information that includes the evaluation result of the patient's adverse event is not sent, Step S34 is omitted from the notification process.

Next, as shown in FIG. 4, the medical support apparatus 10 distinguishes whether a doctor's action is necessary or not (Step S35). The process of distinguishing whether the doctor's action is necessary or not is realized by the determination function 101d in the processing circuit 101. Specifically, the medical support apparatus 10 distinguishes a necessity of the doctor's action based on the evaluation result of the patient's adverse event derived at Step S27. Then, when distinguishing that the doctor's action is unnecessary (Step S35: No), the medical support apparatus 10 determines how to handle the patient without notifying the handling request to the doctor system as shown in FIG. 4 (Step S37). The doctor's action being unnecessary, for instance, refers to a case where the adverse event is identified and the symptoms were mild etc. The process of determining how to handle the patient is realized by the determination function 101d in the processing circuit 101. Specifically, the medical support apparatus 10 determines how to handle the patient such as a necessity of examination or a necessity of prescribing a medicine based on the evaluation result of the patient's adverse event derived at Step S27, without notifying the patient's handling request to the doctor by the doctor system function 201.

Next, as shown in FIG. 4, the medical support apparatus 10 generates an inquiry result information (Step S39). The process of generating the inquiry result information is realized by the determination function 101d in the processing circuit 101. Specifically, the medical support apparatus 10 generates the inquiry result information based on the evaluation result of the patient's adverse event evaluated at Step S27 and how to handle the patient determined at Step S37. The inquiry result information refers to information related to a result of inquiring the patient condition, and includes information such as the adverse event, the severity of symptoms, or an action the patient takes against the adverse event. Note that the inquiry result information may include other information. That is to say, the configuration of the inquiry result information is arbitrary, and for instance, may include information related to the treatment causing the adverse event.

Next, as shown in FIG. 4, the medical support apparatus 10 stores the inquiry result information, the questions, and the patient's response (Step S41). The process of storing the inquiry result information, the questions, and the patient's response is realized by the determination function 101d in the processing circuit 101. Specifically, the medical support apparatus 10 stores to the memory circuit 107 the inquiry result information generated at Step S39, the questions for the patient supplied by sending to the patient terminal 30 at Step S23 and/or Step S33, and the interview result information, which is the patient's response result, as the patient's response received by receiving at Step S25.

Next, as shown in FIG. 4, the medical support apparatus 10 notifies the inquiry result information to the patient terminal 30 (Step S43). The process of notifying the inquiry result information to the patient terminal 30 is realized by the determination function 101d in the processing circuit 101. Specifically, the medical support apparatus 10 sends the inquiry result information via the communication circuit 109 and notifies the inquiry result information to the patient terminal 30. The medical support apparatus 10 ends the notification process when the inquiry result information is notified to the patient terminal 30.

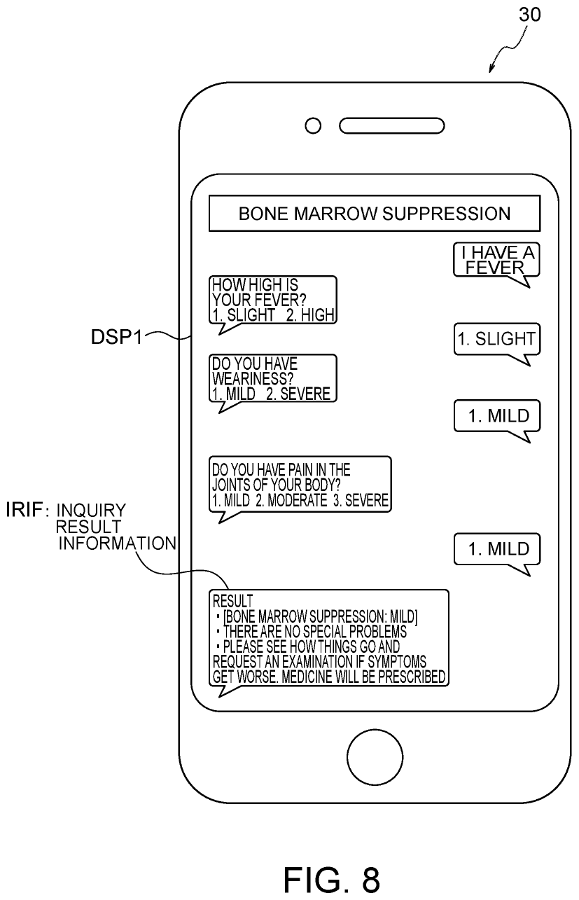

FIG. 8 is a diagram that illustrates an exemplary display status of the display DSP1 of the patient terminal 30 for which the inquiry result information IRIF is notified according to the present embodiment. As shown in FIG. 8, the questions related to the adverse event predicted for the patient supplied by the medical support apparatus 10, the patient's response to the questions, information related to the adverse event, and the inquiry result information IRIF notified at Step S43 are displayed on the display DSP1 of the patient terminal 30. More specifically, as shown in FIG. 8, bone marrow suppression is displayed as the adverse event on the upper portion of the display DSP1, the patient's response input by the patient via the patient terminal 30 is displayed on the right side of the display DSP1, and the questions related to the adverse event predicted for the patient sent by the medical support apparatus 10 and the inquiry result information IRIF are displayed on the left side of the display DSP1. As shown in FIG. 8, for instance, “Bone marrow suppression: mild” and “There are no special problems” which are the evaluation result of the patient's adverse event are displayed as the inquiry result information IRIF, as well as “Please see how things go and request an examination if symptoms get worse. Medicine will be prescribed,” as how to handle the patient.

Note that the inquiry result information IRIF may include other information. That is to say, the configuration of the inquiry result information IRIF is arbitrary. For instance, the inquiry result information IRIF may include the patient's medical information, such as radiotherapy to head and neck, as a reason why the adverse event occurred.

On the other hand, when distinguishing that the doctor's action is necessary based on the evaluation result of the patient condition at Step S35 (Step S35: Yes), the medical support apparatus 10 generates a patient condition information as shown in FIG. 5 (Step S45). The doctor's action being necessary, for instance, refers to a case where the adverse event is identified and the symptoms are moderate or severe, or a case where the adverse event cannot be identified. The process of generating the patient condition information at Step S45 is realized by the determination function 101d in the processing circuit 101. Specifically, the medical support apparatus 10 generates the patient condition information based on the evaluation result of the patient's adverse event derived at Step S27, or the interview result information, which is the patient's response result to the questions received at Step S25, and/or the patient's medical information.

The patient condition information refers to information related to the patient condition, and if the adverse event is identified based on the evaluation result of the patient's adverse event derived at Step S27, the patient condition information includes information related to the adverse event and the severity of symptoms based on the evaluation result of the patient's adverse event derived at Step S27. Also, if the adverse event is not identified based on the evaluation result of the patient's adverse event derived at Step S27, the patient condition information includes the information related to the patient's symptoms based on the interview result information, which is the patient's response result, and the patient's medical information etc. The patient's medical information included in the patient condition information, for instance, refers to the patient's medical care information such as a medical care record related to the information related to the patient's symptoms. Note that the patient condition information may include other information. That is to say, the configuration of the patient condition information is arbitrary, and may include information such as the questions related to the adverse event predicted for the patient, the interview result information, which is the patient's response result to the questions, and the candidates of the adverse event if the adverse event is not identified.

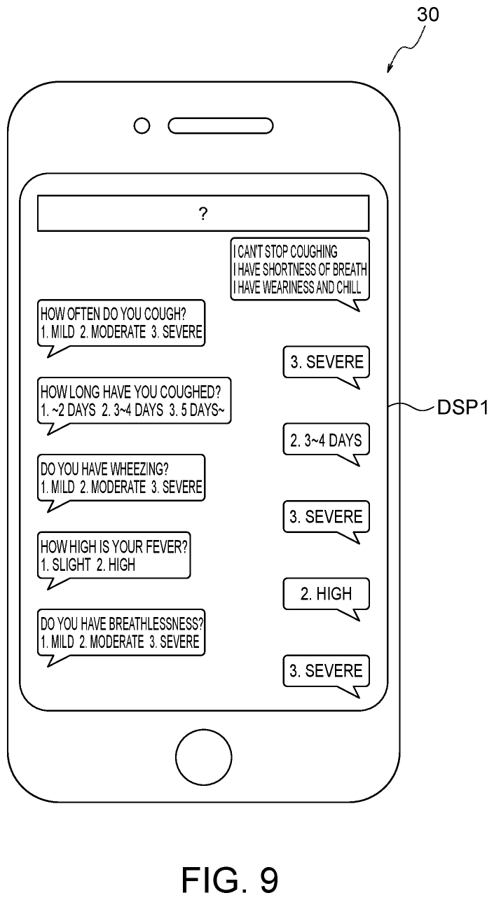

FIG. 9 is a diagram that illustrates an exemplary display status of the display DSP1 of the patient terminal 30 when the adverse event cannot be identified according to the present embodiment. As shown in FIG. 9, the display DSP1 of the patient terminal 30 displays the questions related to the adverse event predicted for the patient supplied by the medical support apparatus 10 and the patient's response to the questions. Likewise, as shown in FIG. 9, if the adverse event cannot be identified based on the evaluation result of the patient's adverse event derived at Step S27, for instance, a sign indicating that there is no corresponding adverse event is displayed on the upper portion of the display DSP1 of the patient terminal 30.

Next, as shown in FIG. 5, the medical support apparatus 10 notifies the handling request to the doctor system (Step S47). The process of notifying the handling request to the doctor system is realized by the determination function 101d in the processing circuit 101. Specifically, the determination function 101d sends and notifies to the doctor system function 201 the handling request indicating that the doctor's action is necessary along with the patient condition information generated at Step S45 based on the evaluation result of the patient's adverse event derived at Step S27. The doctor system function 201 displays to the display 105 the handling request indicating that the doctor's action is necessary, along with the patient condition information generated at Step S45.

Note that, at Step S47, the determination function 101d sends and notifies to the doctor system function 201 the handling request etc. indicating that the doctor's action is necessary, but the destination of the handling request is not limited to this. That is to say, the destination of the handling request etc. is arbitrary, and for instance, if one or more doctor systems are connected to the medical support apparatus 10 in a communicative manner, the determination function 101d may send and notify the handling request indicating that the doctor's action is necessary along with the patient condition information generated at Step S45 to the doctor system of a professional doctor who has knowledge about the patient's symptoms included in the patient condition information based on the patient condition information, or may send and notify the handling request indicating that the doctor's action is necessary along with the patient condition information generated at Step S45 to both the doctor system of the professional doctor and the doctor system function 201. Likewise, the doctor system 201 displays the handling request to the display 105, but embodiments are not limited to this. The determination function 101d may display the handling request to the display 105 and notify.

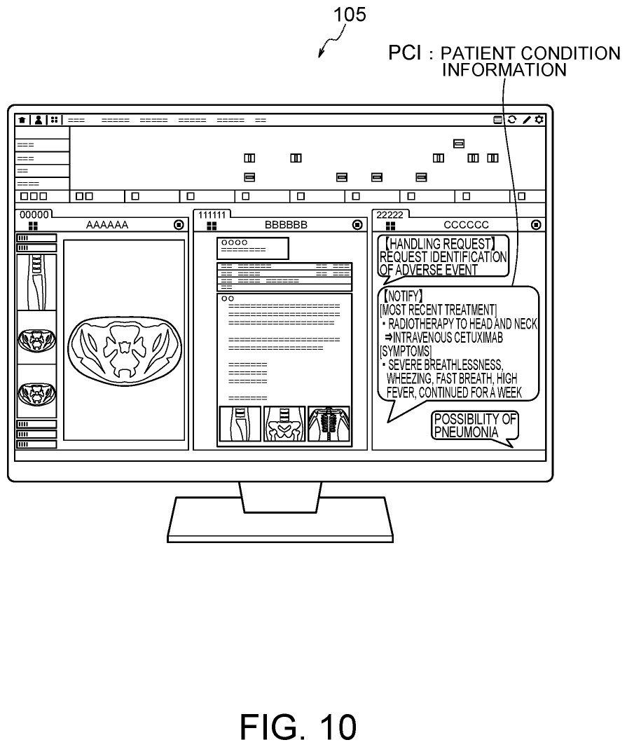

FIG. 10 is a diagram that illustrates an exemplary display status of the display 105 of the medical support apparatus 10 according to the present embodiment. As shown in FIG. 10, “Request identification of the adverse event” as the handling request, information indicating a change from radiotherapy to head and neck to intravenous cetuximab in the most recent treatment as the patient condition information PCI, and information related to the patient symptoms based on the interview result information, which is the patient's response result received at Step S25 are displayed on the display 105 of the medical support apparatus 10. Likewise, “Possibility of pneumonia” is displayed on the display 105 of the medical support apparatus 10 as the doctor's response to the handling request.

Next, as shown in FIG. 5, the medical support apparatus 10 distinguishes whether the doctor's response to the handling request has been received or not (Step S49). The process of distinguishing whether the doctor's response has been received or not is realized by the determination function 101d in the processing circuit 101. If the doctor's response to the handling request at Step S49 has not been received (Step S49: No), the determination function 101d repeats and standbys the process of Step S49 until receiving the doctor's response to the handling request. Here, to receive the doctor's response, for instance, refers to the doctor system function 201 receiving the electrical signal of the input operation from the doctor via the input interface 103 and the determination function 101d receiving the doctor's response based on the electrical signal of the input operation from the doctor. Note that the determination function 101d may receive the electrical signal of the input operation from the doctor via the input interface 103 to receive the doctor's response based on the electrical signal of the input operation from the doctor.

On the other hand, as shown in FIG. 5, if the determination function 101d receives the doctor's response to the handling request at Step S49 (Step S49: Yes), the medical support apparatus 10 determines how to handle the patient (Step S51). Specifically, the medical support apparatus 10 determines how to handle the patient based on the doctor's response received at Step S49. For instance, when “ Possibility of pneumonia” has been received as the doctor's response as shown in FIG. 10, the medical support apparatus 10 determines how to handle the patient such as the examination of the doctor against the possibility of pneumonia or request the doctor for prescription of medicine.

Next, as shown in FIG. 5, the medical support apparatus 10 generates the inquiry result information IRIF (Step S53). The process of generating the inquiry result information IRIF is realized by the determination function 101d in the processing circuit 101. Specifically, the medical support apparatus 10 generates the inquiry result information IRIF based on the doctor's response to the handling request received at Step S49 and how to handle the patient determined at Step S51. Note that if the adverse event is identified based on the evaluation result of the patient's adverse event derived at Step S27, the medical support apparatus 10 may generate the inquiry result information IRIF based on the evaluation result of the patient's adverse event derived at Step S27 and how to handle the patient determined at Step S51 instead of the doctor's response to the handling request received at Step S49.

Next, as shown in FIG. 5, the medical support apparatus 10 stores the inquiry result information IRIF, the questions, and the patient's response (Step S55). The process of storing the inquiry result information IRIF, the questions, and the patient's response is realized by the determination function 101d in the processing circuit 101. Specifically, the medical support apparatus 10 stores the inquiry result information IRIF generated at Step S53, the questions related to the adverse event predicted for the patient supplied by sending to the patient terminal 30 at Step S23 and/or Step S33, and the interview result information, which is the patient's response result received at Step S25.

Next, as shown in FIG. 5, the medical support apparatus 10 notifies the inquiry result information IRIF to the patient terminal 30 (Step S57). The process of notifying the inquiry result information IRIF to the patient terminal 30 is realized by the determination function 101d in the processing circuit 101. Specifically, the medical support apparatus 10 sends the inquiry result information IRIF generated at Step S53 via the communication circuit 109 and notifies to the patient terminal 30.

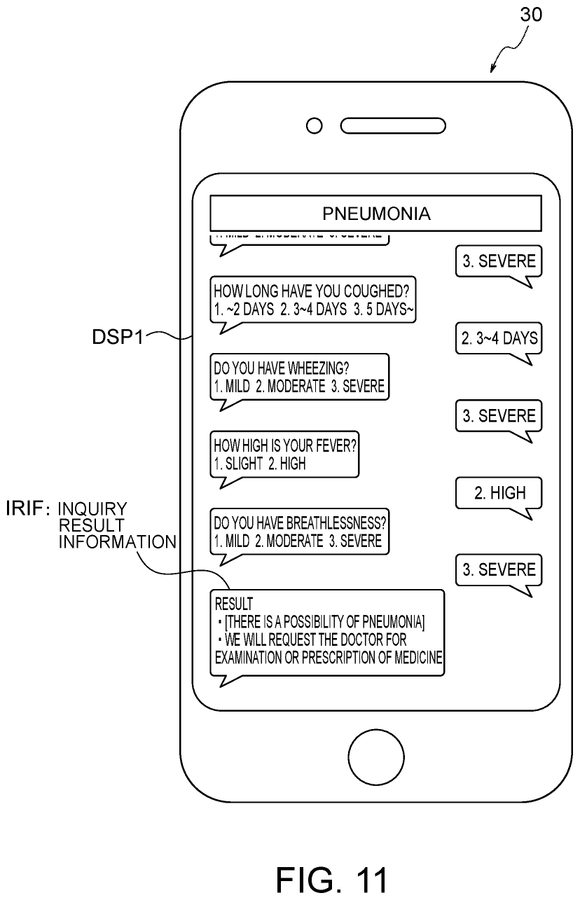

FIG. 11 is a diagram that illustrates an exemplary display status of the display DSP1 of the patient terminal 30 for which the inquiry result information IRIF is notified according to the present embodiment. As shown in FIG. 11, the questions related to the adverse event predicted for the patient supplied by the medical support apparatus 10, the patient's response to the questions related to the adverse event predicted for the patient, the adverse event, and the inquiry result information IRIF notified at Step S57 are displayed on the display DSP1 of the patient terminal 30. More specifically, as shown in FIG. 11, pneumonia is displayed as the adverse event on the upper portion of the display DSP1, the patient's response etc. which the patient has inputted via the patient terminal 30 is displayed on the right side of the display DSP1, and the questions for the patient and the inquiry result information IRIF sent by the medical support apparatus 10 are displayed on the left side of the display DSP1. As shown in FIG. 11, “There is a possibility of pneumonia” which is the doctor's response and “We will request the doctor for examination or prescription of medicine” which is how to handle the patient, are displayed on the display DSP1 of the patient terminal 30 as the inquiry result information IRIF.

The notification process according to the present embodiment ends by notifying the inquiry result information IRIF to the patient terminal 30 at Step S57.

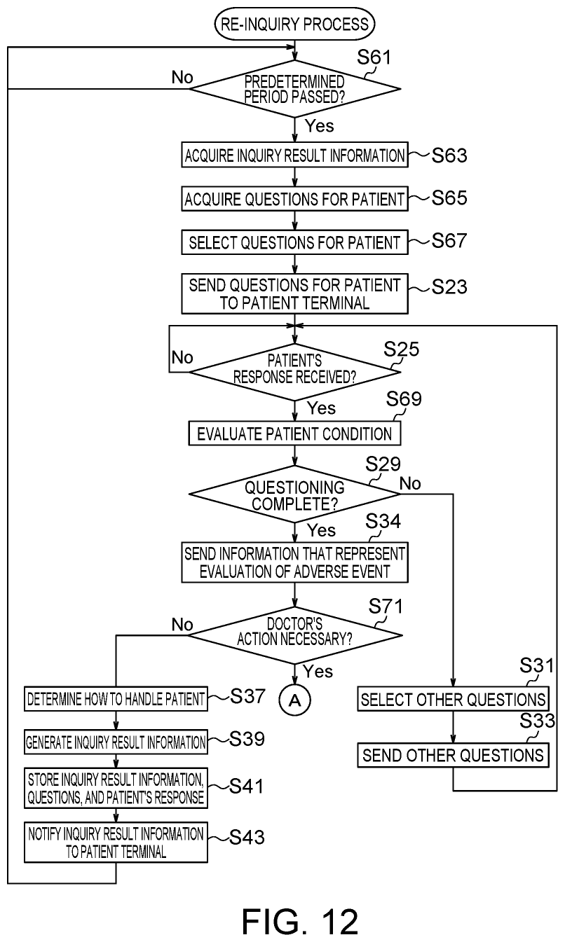

FIG. 12 is a flowchart that describes a content of a re-inquiry process executed in the medical support apparatus 10 according to the present embodiment. In the re-inquiry process, the medical support apparatus 10 reacquires the interview information, resupplies the interview to patient terminal 30, re-inquires the patient based on the inquiry result information IRIF after the predetermined period has passed, re-evaluates the patient's adverse event based on the interview result information, which is the patient's response result, or determines how to handle the patient based on the re-evaluation result of the patient's adverse event and notifies to the patient terminal 30. For instance, the re-inquiry process refers to a process executed when the predetermined period has passed from the previous response to the patient or the change of the treatment plan.

As shown in FIG. 12, the medical support apparatus 10 first distinguishes whether the predetermined period has passed from the previous response to patient or the change of the treatment plan (Step S61). The process of distinguishing whether the predetermined period has passed is realized by the interview information supply function 101b in the processing circuit 101. Specifically, if the predetermined period has not passed from the notification of the inquiry result information IRIF, which is the previous response to the patient, or the change of the treatment plan, i.e., when the predetermined time has not arrived from the notification of the inquiry result information IRIF, which is the previous response to the patient, or the change of the treatment plan (Step S61: No), the medical support apparatus 10 repeats the process of Step S61 and standbys until the predetermined period has passed from the notification of the inquiry result information IRIF, which is the previous response to the patient, or the change of the treatment plan.

On the other hand, if the predetermined period has passed from the notification of the inquiry result information IRIF, which is the previous response to the patient, or the change of the treatment plan, i.e., when the predetermined time has arrived from the notification of the inquiry result information IRIF, which is the previous response to the patient, or the change of the treatment plan at Step S61 (Step S61: Yes), as shown in FIG. 12, the medical support apparatus 10 acquires the inquiry result information IRIF (Step S63). The process of acquiring the inquiry result information IRIF is realized by the medical information acquisition function 101a in the processing circuit 101. Specifically, the medical support apparatus 10 acquires the inquiry result information IRIF generated in the notification process from the memory circuit 107. Note that the medical support apparatus 10 may acquire the patient's medical information from the memory circuit 107 along with the inquiry result information IRIF.

Next, as shown in FIG. 12, the medical support apparatus 10 acquires the questions for the patient (Step S65). The process of acquiring the questions for the patient is realized by the interview information supply function 101b in the processing circuit 101. Specifically, the medical support apparatus 10 acquires the interview information related to the adverse event predicted for the patient as the questions for the patient based on the inquiry result information IRIF from the question list LT1 stored in the memory circuit 107.

Next, as shown in FIG. 12, the medical support apparatus 10 selects the questions for the patient (Step S67). The process of selecting the questions for the patient is realized by the interview information supply function 101b in the processing circuit 101. Specifically, the medical support apparatus 10 selects the questions from the questions related to the adverse event predicted for the patient acquired at Step S65. For instance, if the adverse event is bone marrow based on the inquiry result information IRIF, the medical support apparatus 10 selects the question “How high is your fever?” which is the first question for bone marrow. Since the process of Step S23 and Step S25 after Step S67 are similar to the process of the notification process described above, description will be omitted.

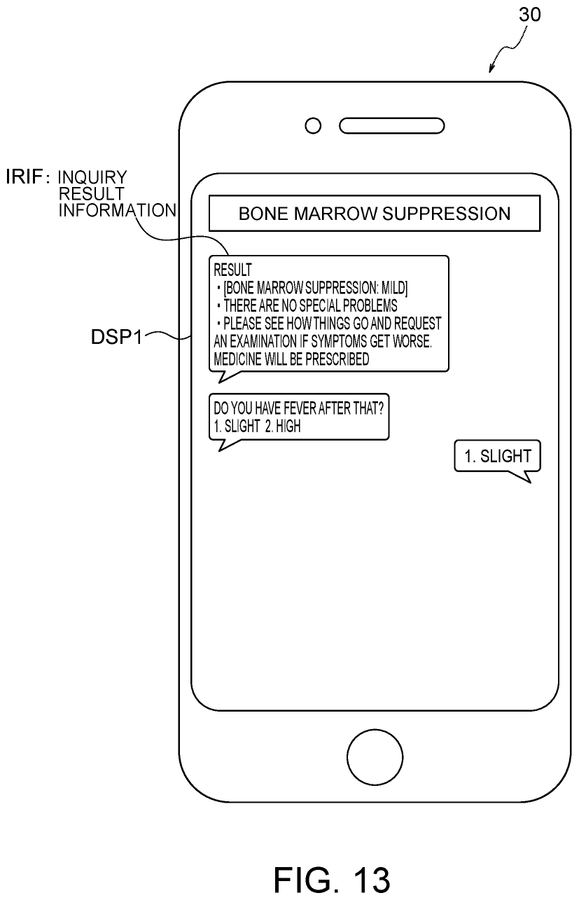

FIG. 13 is a diagram that illustrates an exemplary display status of the display DSP1 of the patient terminal 30 according to the present embodiment. As shown in FIG. 13, the question related to the adverse event predicted for the patient supplied by the medical support apparatus 10, the adverse event, and the response input by the patient via the patient terminal 30 are displayed on the display DSP1 of the patient terminal 30. More specifically, as shown in FIG. 13, bone marrow suppression is displayed as the adverse event on the upper portion of the display DSP1, “Do you have fever after that? 1. mild 2. severe” is displayed as the question related to the adverse event predicted for the patient supplied by the medical support apparatus 10 on the left side of the display DSP1, and “1. mild,” which is the response input by the patient via the patient terminal 30, is displayed on the right side of the display DSP1. Note that, as shown in FIG. 13, the patient terminal 30 may display the previous inquiry result information IRIF supplied by the medical support apparatus 10. That is to say, the medical support apparatus 10 may send the previous inquiry result information IRIF to the patient terminal 30.

Next, as shown in FIG. 12, the medical support apparatus 10 evaluates the patient condition (Step S69). The process of evaluating the patient condition is realized by the evaluation function 101c in the processing circuit 101. Specifically, the medical support apparatus 10 reevaluates the patient's adverse event according to the patient condition based on the interview result information, which is the patient's response result received at Step S25 of the re-inquiry process. More specifically, the medical support apparatus 10 identifies the severity of symptoms based on the interview result information, which is the patient's response, reevaluates the patient's adverse event, and rederives the evaluation result of the patient's adverse event based on the inquiry result information IRIF and the interview result information, which is the patient's response result.

Note that such evaluation result of the patient's adverse event may include the evaluation comment whether the symptoms have aggravated or improved etc., which is the result of comparing the severity of symptoms included in the interview result information IRIF acquired at Step S63 and the severity of symptoms identified at Step S69, based on the inquiry result information IRIF acquired at Step S63 and the interview result information IRIF, which is the patient's response result. Since the process of Step S29, Step S31, and Step S33 after Step S69 are similar to the process of the notification process described above, description will be omitted.

On the other hand, if the severity of symptoms is identified based on the evaluation result of the patient's adverse event derived at Step S69 and the questioning is complete at Step S29 of the re-inquiry process (Step S29: Yes), the medical support apparatus 10 sends the information that represents the evaluation result of the adverse event (Step S34). Note that, even when the severity of symptoms is identified at Step S29 and the questions acquired at Step S65 are left, the medical support apparatus 10 may complete the questioning without questioning the rest of the questions. Since the process of Step S34 is similar to that of the notification process described above, description will be omitted.

Next, as shown in FIG. 12, the medical support apparatus 10 distinguishes whether the doctor's action is necessary or not (Step S71). The process of distinguishing whether the doctor's action in necessary or not is realized by the determination function 101d in the processing circuit 101. Specifically, the medical support apparatus 10 distinguishes the necessity of the doctor's action based on the evaluation result of the patient's adverse event derived at Step S69 and the inquiry result information IRIF acquired at Step S61. Then, when distinguishing that the doctor's action is unnecessary (Step S71: No), the medical support apparatus 10 determines how to handle the patient without notifying the handling request to the doctor by the doctor system function 201 as shown in FIG. 4 (Step S37). The doctor's action being unnecessary refers to a case such as where the severity of symptoms related to the adverse event improves based on the evaluation result of the patient's adverse event derived at step S69 and the inquiry result information IRIF acquired at Step S61. Since the process of Step S39, Step S41, and Step S43 after Step S37 is similar to the process of the notification process described above, description will be omitted. After the process of Step S43, the medical support apparatus 10 repeats the process of Step S61 and standbys until the predetermined period passes.

On the other hand, when distinguishing that the doctor's action is necessary based on the evaluation result of the patient condition derived at Step S69 and the inquiry result information IRIF acquired at Step S61 at Step S71 (Step S71: Yes), the medical support apparatus 10 generates the patient condition information PCI as shown in FIG. 5 (Step S45). The doctor's action being necessary refers to a case such as where the severity of symptoms related to the adverse event gets worse or does not improve based on the evaluation result of the patient's adverse event derived at step S69 and the inquiry result information IRIF acquired at Step S61. Since the process of Step S47, Step S49, Step S51, Step S53, Step S55, and Step S57 after Step S45 is similar to the process of the notification process described above, description will be omitted.

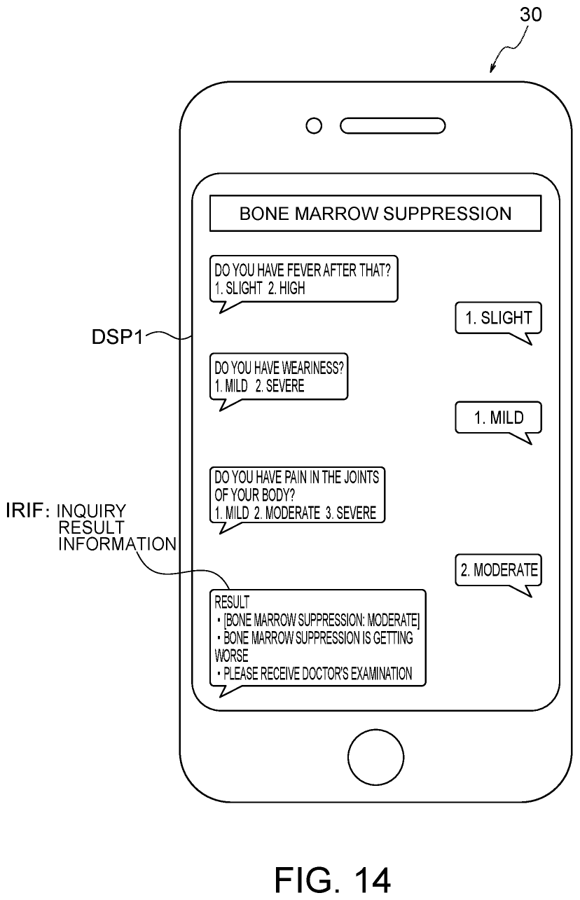

FIG. 14 is a diagram that illustrates an exemplary display status of the display DSP1 of the patient terminal 30 where the inquiry result information IRIF is notified according to the present embodiment. As shown in FIG. 14, the questions for the patient supplied by the medical support apparatus 10, the patient's response to the questions, information related to the adverse event, and the inquiry result information IRIF notified at Step S57 are displayed on the display DSP1 of the patient terminal 30. As shown in FIG. 14, for instance, “Bone marrow suppression: moderate”, “Bone marrow suppression is getting worse”, which are the evaluation result of the patient's adverse event, are displayed as the inquiry result information IRIF, as well as “Please receive doctor's examination.” as how to handle the patient.

The re-inquiry process according to the present embodiment ends by sending the inquiry result information IRIF to the patient terminal 30 at Step S57.

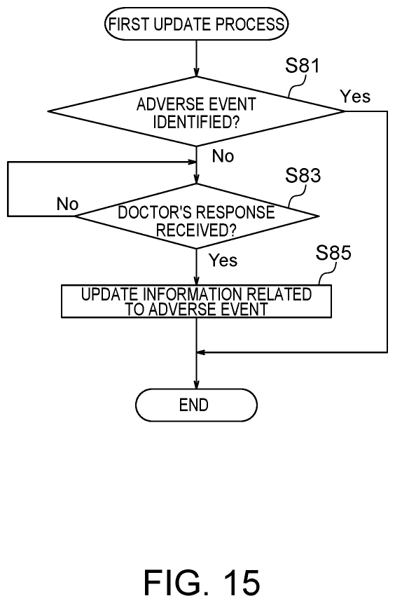

FIG. 15 is a flowchart that describes a content of a first update process executed in the medical support apparatus 10 according to the present embodiment. In the first update process, the medical support apparatus 10 distinguishes whether the adverse event is identified, receives the doctor's response if the adverse event cannot be identified, and updates the information related to the adverse event based on the received doctor's response. For instance, the first update process refers to a process executed when the evaluation result of the patient's adverse event according to the patient condition is derived.

As shown in FIG. 15, the medical support apparatus 10 first distinguishes whether the adverse event is identified or not (Step S81). The process of distinguishing whether the adverse event is identified or not is realized by the first update function 101e in the processing circuit 101. Specifically, when the medical support apparatus 10 identifies the adverse event based on the evaluation result of the patient's adverse event derived at Step S27 of the notification process described above (Step S81: Yes), the first update process according to the present embodiment ends.

On the other hand, when the adverse event is not identified based on the evaluation result of the patient's adverse event derived at Step S27 of the notification process described above at Step S81 (Step S81: No), the medical support apparatus 10 distinguishes whether the doctor's response has been received or not (Step S83). The process of distinguishing whether the doctor's response to the handling request has been received or not is realized by the first update function 101e in the processing circuit 101. Specifically, when the doctor's response to the handling request has not been received (Step S83: No), the medical support apparatus 10 repeats the process of Step S83 and standbys until receiving the doctor's response to the handling request.

On the other hand, when the doctor's response to the handling request has been received at Step S83 (Step S83: Yes), the medical support apparatus 10 updates information related to the adverse event stored in the memory circuit 107 as shown in FIG. 15 (Step S85). The process of updating the information related to the adverse event is realized by the first update function 101e in the processing circuit 101. Specifically, the medical support apparatus 10 updates the information related to the adverse event stored in the memory circuit 107 based on the doctor's response received at Step S83. For instance, if “Possibility of pneumonia” is received as the doctor's response, the adverse event “pneumonia” and information related to symptoms of pneumonia are associated, and the information related to the adverse event stored at the memory circuit 107 is updated.

The first update process according to the present embodiment ends by updating the adverse event at Step S85.

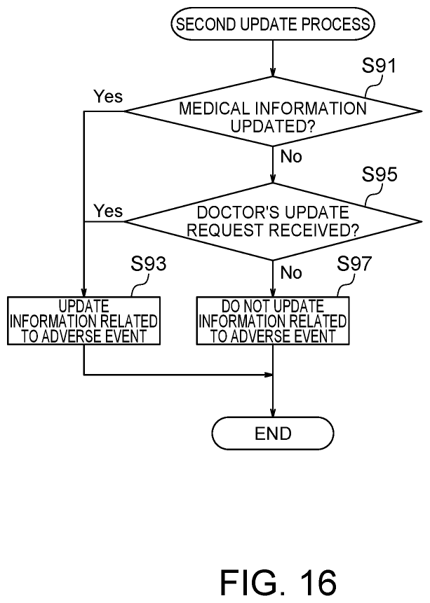

FIG. 16 is a flowchart that describes a content of a second update process executed in the medical support apparatus 10 according to the present embodiment. In the second update process, the medical support apparatus 10 distinguishes whether there is an update of the medical information or a doctor's update request, and updates the information related to the adverse event if there is the update of either the medical information or the doctor's update request. For instance, the second update process refers to a process executed when there is the update of the medical information or the doctor's update request.

As shown in FIG. 16, the medical support apparatus 10 first distinguishes whether the patient's medical information is updated or not (Step S91). The process of distinguishing whether the patient's medical information is updated or not is realized by the second update function 101f in the processing circuit 101. when the patient's medical information such as an inspection result or a course of treatment is updated at Step S91 (Step S91:Yes), the medical support apparatus 10 updates the information related to the adverse event stored in the memory circuit 107 based on the updated patient's medical information as shown in FIG. 16 (Step S93). More specifically, the medical support apparatus 10, for instance, updates so as to delete an item related to digestive insufficiency from the information related to the adverse event stored in the memory circuit 107 based on a result that digestive organs have no problem as the inspection result. Note that, when information such as the inspection result and the course of treatment is updated as the patient's medical information, the medical support apparatus 10 may update so as to associate and add the adverse event and information related to the symptoms concerning the adverse event to the information related to the adverse event stored in the memory circuit 107 based on the updated patient's medical information.

On the other hand, when the patient's medical information is not updated at Step S91 (Step S91:No), the medical support apparatus 10 distinguishes whether the doctor's update request has been received or not as shown in FIG. 16 (Step S95). The process of distinguishing whether the doctor's update request has been received or not is realized by the second update function 101f in the processing circuit 101. When the doctor's update request is received at Step S91 (Step S95: Yes), the medical support apparatus 10 updates information related to the adverse event based on the received doctor's update request as shown in FIG. 16 (Step S93). More specifically, for instance, when receiving the doctor's update request to add pneumonia to the information related to the adverse event, the medical support apparatus 10 associates the adverse event “pneumonia” and the symptoms of “pneumonia”, and updates information related to the adverse event stored in the memory circuit 107.

Here, receiving the doctor's update request, for instance, refers to the doctor system function 201 receiving the electrical signal of the input operation received from the doctor via the input interface 103 and the second update function 101f receiving the doctor's update request based on the electrical signal of the input operation received from the doctor. Note that the second update function 101f may receive the electrical signal of the input operation received from the doctor via the input interface 103 and receive the doctor's update request based on the electrical signal of the input operation received from the doctor.

On the other hand, when the doctor's update request has not been received at Step S95 (Step S95: No), the medical support apparatus 10 does not update information related to the adverse event as shown in FIG. 16 (Step S97).

The second update process ends by updating information related to the adverse event at Step S93 or not updating information related to the adverse event at Step S97.

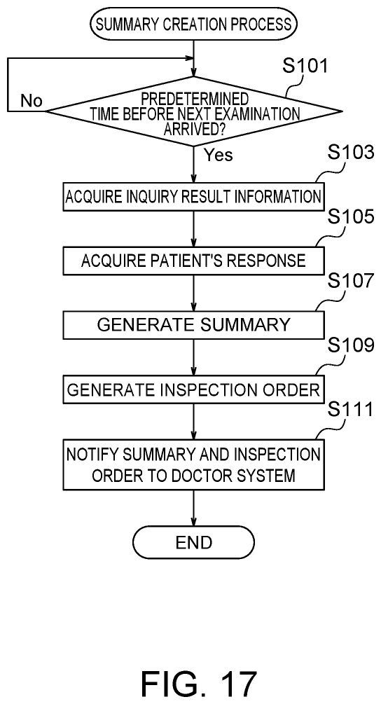

FIG. 17 is a flowchart that describes a content of a summary creation process executed in the medical support apparatus 10 according to the present embodiment. The summary creation process refers to a process that creates a summary that summarizes information related to the patient condition by acquiring the one or more inquiry result information IRIF stored in the memory circuit 107 and the interview result information, which is the patient's response result to the questions, when the predetermined period before a next examination arrives. For instance, the summary creation process refers to a process executed when the predetermined period before the next examination arrives.

As shown in FIG. 17, the medical support apparatus 10 first distinguishes whether the predetermined period before the next examination has arrived (Step S101). The process of distinguishing whether the predetermined period before the next examination has arrived is realized by the summary creation function 101g in the processing circuit 101. When the predetermined period before the next examination has not arrived at Step S101 (Step S101: No), the medical support apparatus 10 repeats Step S101 and standbys until the predetermined period before the next examination arrives.

On the other hand, when the predetermined period before the next examination has arrived at Step S101 (Step S101: Yes), the medical support apparatus 10 acquires the inquiry result information IRIF as shown in FIG. 10 (Step S103). The process of acquiring the inquiry result information IRIF is realized by the summary creation function 101g in the processing circuit 101. Specifically, the medical support apparatus 10 acquires from the memory circuit 107 the one or more inquiry result information IRIF generated in the notification process or the re-inquiry process described above.

Next, the medical support apparatus 10 acquires the patient's response as shown in FIG. 17 (Step S105). The process of acquiring the patient's response is realized by the summary creation function 101g in the processing circuit 101. Specifically, the medical support apparatus 10 acquires the interview result information from the memory circuit 107 as the patient's response to the questions related to the adverse event predicted for the patient received in the notification process or the re-inquiry process described above.

Next the medical support apparatus 10 creates the summary as shown in FIG. 17 (Step S107). The process of creating the summary is realized by the summary creation function 101g in the processing circuit 101. Specifically, the medical support apparatus 10 creates the summary related to the patient condition based on the inquiry result information IRIF acquired at Step S103 and the interview result information, which is the patient's response result acquired at Step S105. The summary refers to information summarizing information such as the adverse event based on the inquiry result information IRIF, the patient symptoms related to the adverse event, or the severity of symptoms etc. Note that the summary may include other information. That is to say, the configuration of the summary is arbitrary. the summary, for instance, may include information such as a patient ID or the prescribed medicine.

Next, the medical support apparatus 10 generates an inspection order information as shown in FIG. 17 (Step S109). The process of generating the inspection order information is realized by the summary creation function 101g in the processing circuit 101. Specifically, the medical support apparatus 10 generates an inspection order information to request the doctor for the necessary inspection order based on the inquiry result information IRIF acquired at Step S103 and the interview result information, which is the patient's response result acquired at Step S105.

The inspection order information refers to information when ordering a laboratory technician for a necessary image inspection. At least information related to an inspection target is included in the inspection information. Note that the inspection order information may include the summary. That is to say, at Step S107, the inspection order information may be generated along with generating the summary. Step S109 is omitted when the inspection order information is generated at Step S107. Likewise, the medical support apparatus 10 may not generate the inspection order information. Step S109 is omitted if the inspection order information is not generated.

Next, the summary creation function 101g notifies the summary and the inspection order information to the doctor system as shown in FIG. 17 (Step S111). The process of notifying the summary and the inspection order information to the doctor system is realized by the summary creation function 101g in the processing circuit 101. Specifically, the summary creation function 101g sends and notifies the summary and the inspection order information to the doctor system function 201. The doctor system function 201 displays the summary and the inspection order information on the display 105. Note that the doctor system function 201 displays the summary and the inspection order information on the display 105, but embodiments are not limited to this. The summary creation function 101g may display and notify the summary and the inspection order information on the display 105.

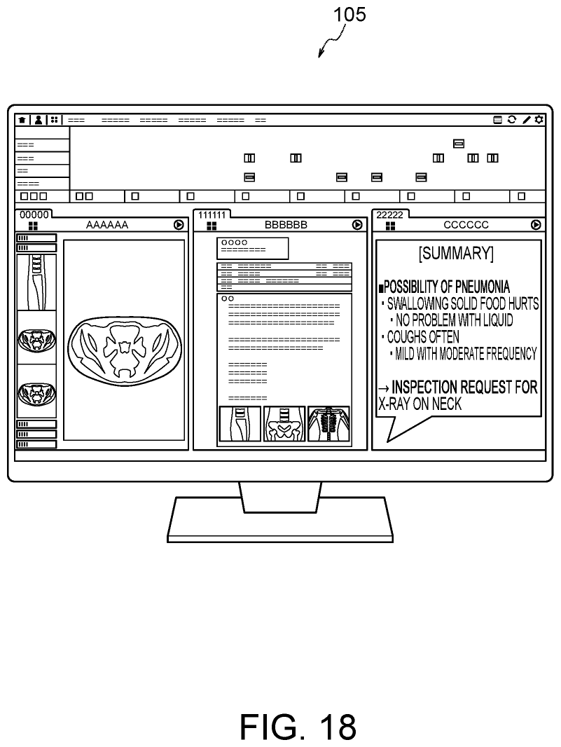

FIG. 18 is a diagram that illustrates an exemplary display status of the summary and the inspection order information displayed on the display 105 of the medical support apparatus 10 according to the present embodiment. As shown in FIG. 18, “Possibility of pneumonia, Swallowing solid food hurts, No problem with liquid, Coughs often, Mild with moderate frequency” is displayed as the summary on the display 105 of the medical support apparatus 10. “Inspection request for X-ray on neck” is also displayed as the inspection order information included in the summary.

The summary creation process according to the present embodiment ends by notifying the summary and the inspection order information to the doctor system at Step S111.

In the medical support system 1 according to the present embodiment as described above, since the medical support apparatus 10 acquires the interview information related to the adverse event predicted for the patient, supplies the acquired interview information to the patient terminal 30, receives the interview result information for the supplied interview information, and evaluates the patient's adverse event based on the received interview result information, a treatment prognosis follow-up may be performed without increasing a doctor's workload.

Likewise, since the medical support apparatus 10 supplies to the doctor system the evaluation result of the patient's adverse event as information that represents the evaluation result of the patient's adverse event, the doctor may timely respond to a patient's incident outside the hospital in response to information that represents the evaluation result of the patient's adverse event without being compelled to respond to a patient's incident outside the hospital each time. That is to say, the doctor may diagnose the patient efficiently and appropriately.

Furthermore, since the medical support apparatus 10 supplies to the patient terminal 30 the evaluation result of the patient's adverse event as information that represents the evaluation result of the patient's adverse event, the patient may receive the response which reflects the patient's own course of treatment from the medical support apparatus 10 and receive appropriate diagnosis from the doctor if the doctor's action is necessary.

Likewise, since the medical support apparatus 10 acquires the interview information based on the patient's medical information, supplies the interview information related to the adverse event predicted for the patient, evaluates the patient's adverse event based on the patient's response result to the interview information, and determine how to handle the patient based on the evaluation result of the patient's adverse event, it is possible to make the response that reflects the patient's medical information. As a result, the doctor is not compelled to respond to the patient's incident outside the hospital each time, and the doctor's workload may be minimally suppressed. That is to say, the doctor may diagnose the patient efficiently and appropriately.

Likewise, since the medical support apparatus 10 distinguishes the necessity of the doctor's action based on the evaluation result of the patient's adverse event, the doctor may respond to the patient's incident outside the hospital while minimally suppressing the increase of the doctor's workload. That is to say, if the doctor's action is unnecessary, the medical support apparatus 10 notifies the response that reflects the patient's medical information to the patient terminal 30, and if the doctor's action is necessary, the medical support apparatus 10 may respond to the patient's incident after notifying the handling request to the doctor system and receiving the doctor's response. As a result, the doctor may diagnose the patient efficiently and appropriately.

Note that the medical support system 1 according to the present application described above may be applied where the medical support apparatus 10 and the doctor system 150 are separate as shown in FIG. 2. When the medical support apparatus 10 and the doctor system 150 are separate, the processing circuit 101 of the medical support apparatus 10 may not comprise the doctor system function 201.

Likewise, since the medical support apparatus 10 and the doctor system 150 are separate, the medical support apparatus 10 sends and notifies the handling request, the patient condition information PCI, the summary, or the inspection order information to the doctor system 150 via the communication circuit 109. For instance, the determination function 101d in the processing circuit 101 sends and notifies the handling request along with the patient condition information PCI to the doctor system 150 via the communication circuit 109 at Step S47 of the notification process and the re-inquiry process. Similarly, the summary creation function 101g in the processing circuit 101 sends and notifies the summary and the inspection order information to the doctor system 150 via the communication circuit 109 at Step S111 of the summary creation process shown in FIG. 17.

Furthermore, since the medical support apparatus 10 and the doctor system 150 are separate, the medical support apparatus 10 receives the doctor's response from the doctor system 150 via the communication circuit 109. For instance, the determination function 101d in the processing circuit 101 receives the doctor's response to the handling request via the communication circuit 109 at Step S49 of the notification process and the re-inquiry process. Similarly, the second update function 101f in the processing circuit 101 receives the doctor's update request via the communication circuit 109 at Step S95 of the second update process shown in FIG. 16.

As described above, even when the medical support apparatus 10 and the doctor system 150 are separate, similar to the case where the medical support apparatus 10 and the doctor system 150 are combined, the medical support apparatus 10 may notify the response that reflects the patient's medical information to the patient terminal 30, and, if the doctor's action is necessary, the medical support apparatus 10 may respond to the patient's incident after notifying the handling request to the doctor system and receiving the doctor's response. As a result, the doctor may diagnose the patient efficiently and appropriately.

Note that the word “processor” used in above descriptions means circuits such as, for example, a Central Processing Unit (CPU), a Graphics Processing Unit (GPU), an Application Specific Integrated Circuit (ASIC), a programmable logic device (for example, a Simple Programmable Logic Apparatus (SPLD), a Complex Programmable Logic Apparatus (CPLD), and a Field Programmable Gate Array (FPGA)). The processor executes functions by reading and executing programs stored in the memory. Note that programs may be configured to be directly integrated in the processor instead of being storing in the memory. In this case, the processor realizes functions by reading and executing programs stored in the circuit. Note that the processor is not limited to the case arranged as a single processor circuit, but may be configured as a single processor by combining a plurality of independent circuits to realize functions. Furthermore, a plurality of component elements may be integrated into one processor to realize the functions.

While certain embodiments have been described, these embodiments have been presented by way of example only and are not intended to limit the scope of the inventions. The embodiments may be in a variety of other forms. Furthermore, various omissions, substitutions and changes may be made without departing from the spirit of the inventions. The embodiments and their modifications are included in the scope and the subject matter of the invention, and at the same time included in the scope of the claimed inventions and their equivalents.

1. A medical support apparatus comprising:
processing circuit configured to
acquire interview information related to an adverse event predicted for a patient and supply the interview information to a patient terminal; and
receive an interview result information, which is a patient's response result to the interview information, and evaluate a patient's adverse event based on the interview result information.

2. The medical support apparatus of claim 1, wherein the processing circuit is further configured to supply information that represents an evaluation result of the patient's adverse event to a doctor system.

3. The medical support apparatus of claim 1, wherein the processing circuit is further configured to supply information that represents an evaluation result of the patient's adverse event to the patient terminal.

4. The medical support apparatus of claim 1, wherein the processing circuit is further configured to:
predict the patient's adverse event based on a patient's medical information stored in a memory, and
acquire interview information related to the predicted patient's adverse event.

5. The medical support apparatus of claim 1, wherein the adverse event is an unfavorable medical event that occurs to a patient.

6. The medical support apparatus of claim 1, wherein the processing circuit is further configured to generate the interview information that narrows down the adverse event and supply the interview information to the patient terminal when there are multiple candidates of the predicted adverse event.

7. The medical support apparatus of claim 1, wherein the processing circuit is further configured to supply the interview information to the patient terminal in response to an arrival of a predetermined time.

8. The medical support apparatus of claim 1, wherein the processing circuit is further configured to supply the interview information to the patient terminal in response to a request from the patient terminal.

9. The medical support apparatus of claim 8, wherein the processing circuit is further configured to supply to the patient terminal the medical information related with the adverse event in response to a time of receiving the request from the patient terminal.

10. The medical support apparatus of claim 1, wherein the processing circuit is further configured to determine how to handle the patient based on an evaluation result of the patient's adverse event.

11. The medical support apparatus of claim 10, wherein the processing circuit is further configured to distinguish a necessity of a doctor's action based on the evaluation result of the patient's adverse event,
when the doctor's action is necessary, notify a handling request to a doctor system, and
when the doctor's action is unnecessary, do not notify the handling request to the doctor system.

12. The medical support apparatus of claim 11, wherein the processing circuit is further configured to:
generate a patient condition information, based on the evaluation result of the patient's adverse event or an interview result information and/or a patient's medical information, and
supply and notify the handling request along with the patient condition information to the doctor system.

13. The medical support apparatus of claim 11, wherein the processing circuit is further configured to, when a doctor's response to the handling request has been received, determine how to handle the patient based on the doctor's response.

14. The medical support apparatus of claim 1, wherein the processing circuit is further configured to:
after supplying information that represents an evaluation result of the patient's adverse event to the patient terminal, in response to an arrival of a predetermined time, reacquire the interview information and resupply the interview information to the patient terminal, and
receive the interview result information about the interview information and re-evaluates the patient's adverse event based on the interview result information.

15. The medical support apparatus of claim 13, wherein the processing circuit is further configured to update information related to the adverse event based on the doctor's response.

16. The medical support apparatus of claim 1, wherein the processing circuit is further configured to update information related to the adverse event based on the patient's medical information or a doctor's update request.

17. The medical support apparatus of claim 16, wherein the processing circuit is further configured to:
distinguish whether the patient's medical information is updated or not, and
when the patient's medical information is updated, update information related to the adverse event based on the patient's medical information.

18. The medical support apparatus of claim 16, wherein the processing circuit is further configured to:
distinguish whether the doctor's update request has been received or not, and
when the doctor's update request has been received, update information related to the adverse event based on the doctor's update request.

19. The medical support apparatus of claim 1, wherein the processing circuit is further configured to create a summary that summarizes information related to the patient condition, based on the evaluation result of the patient's adverse event and an interview result information for the interview information.

20. A medical support system, comprising:
a medical support apparatus comprising a memory and processing circuit, wherein the processing circuit is configured to
acquire an interview information related to an adverse event predicted for a patient and supply the interview information to a patient terminal; and
receive an interview result information, which is an interview result of the patient to the interview information, and evaluate the patient's adverse event based on the interview result information; and
the patient terminal configured to receive interview information from the medical support apparatus and supply the interview result information about the interview information to the medical support apparatus.
